(19) United States
(12) Patent Application Publication
(10) Pub. No.: US 2020/0340968 A1
(43) Pub. Date: Oct. 29, 2020

(54) APPARATUS AND METHOD FOR LOW POWER MEASUREMENT OF A LIQUID-QUALITY PARAMETER

(71) Applicant: BLUE I WATER TECHNOLOGIES LTD, Rosh Ha'ayin (IL)

(72) Inventor: NELSON ZAKINOV, HOLON (IL)

(73) Assignee: BLUE I WATER TECHNOLOGIES LTD, Rosh Ha'ayin (IL)

(21) Appl. No.: 16/607,983

(22) PCT Filed: Apr. 23, 2018
(86) PCT No.: PCT/IB2018/052816

§ 371 Date: Oct. 24, 2019

(30) Foreign Application Priority Data
Apr. 24, 2017 (GB) 1706433.8

(51) Int. Cl. G01N 33/18; G01N 21/25; G01F 15/06; G08B 21/18; G08B 21/12

(52) U.S. Cl. CPC G01N 33/1893; G01N 21/251; G08B 21/12; G08B 21/182; G01F 15/06

(57) ABSTRACT

An apparatus for measuring a water-quality parameter of a liquid sample. The apparatus includes: at least one water-quality parameter sensor selected from the group containing: a chlorine sensor; a turbidity sensor; a conductivity sensor; a pH sensor; a temperature sensor; a pressure sensor; a redox sensor; and a flow sensor; a controller configured to control operation between an active mode, a sleep mode, and a turbo-mode; and an energy source management module associated with the controller. The management module manages voltage in the controller and provides for extended power and low electricity consumption.

### CROSS REFERENCE TO RELATED APPLICATIONS

This application claims priority from UK Patent Application No. GB1706433.8 filed 24 Apr. 2017, entitled “APPARATUS AND METHOD FOR LOW POWER MEASUREMENT OF A LIQUID-QUALITY PARAMETER”, which is incorporated in its entirety herein by reference.

### FIELD OF THE INVENTION

The invention relates to an apparatus and method for monitoring the parameters of liquids (e.g. water quality), in particular, to automated measurement of up to a multitude of water-quality parameters in an energy saving manner.

### BACKGROUND OF THE INVENTION

Drinking water is a potential source of numerous diseases and infections afflicting humans, some of which may even be lethal. Various types of equipment have been developed and are commonly used for the measurement of turbidity, color and chlorine content of liquids. In this regard, US 2016/131,578 (Rachman, et al., 2016 May 12) discloses a system and method for the simultaneous measurement of turbidity, color and chlorine content of a liquid sample, and US 2010/320,095 (Galperin et al. 2010-12-23) discloses a water quality measurement device—both of which are incorporated herein in their entirety).

To substantially reduce the risk of contraction of diseases and infections, drinking water is generally treated with chlorine in water treatment plants prior to distribution for human consumption. The chlorine acts as a disinfectant, killing numerous bacteria and viruses found in water by bonding to, and destroying, their outer surfaces.

Chlorine in the water treatment plant is generally added into water as chlorine gas, sodium hypochlorite and/or chloride dioxide. Monitoring the concentration of chlorine is usually performed both in the plant and in monitoring stations located at various points in a water distribution network. Monitoring is performed to ensure that the chlorine concentration in the drinking water is maintained below a level that may pose a hazard for human consumption, yet above a minimum level necessary to substantially eliminate possible bacteria and viruses.

Furthermore, existing equipment typically requires a relatively significant amount of energy, which makes distributed monitoring difficult.

### SUMMARY OF THE INVENTION

The present invention relates to the measurement of at least one of: turbidity, color and chlorine content, of a liquid sample, such as treated water—in particular wherein an apparatus and method for performing the measurement is operated in an energy savings (low energy) manner.

In accordance with embodiments of one aspect of the present invention there is provided an apparatus for measurement of a liquid-quality parameter, in particular low-energy measurement(s). The apparatus includes: at least one water-quality parameter sensor selected from the group containing: a chlorine sensor; a turbidity sensor; a conductivity sensor; a pH sensor; a temperature sensor; a pressure; a redox sensor; and a flow sensor; a controller configured to control operation of the apparatus between an active mode, when the apparatus is performing measurements; and a sleep mode when the apparatus is in a non-measurement, minimally powered state; an energy source management module operably associated with said controller, wherein said module is configured to manage voltage in said controller and provide for extended power and low electricity consumption.

In some embodiments, the controller is configured to further control operation of the apparatus between said active mode, said sleep mode, and a turbo-mode, which is a mode that is employed in the event that measurement of the water-quality parameter is outside a given range. In some embodiments, the chlorine sensor is configured to measure free chlorine or total chlorine.

In some embodiments, the apparatus is configured so that a plurality of water-quality parameter sensors of said at least one sensor are usable in a single liquid sample.

In some embodiments, the controller is further configured to maintain low power to said at least one sensor so that the sensor does not enter a passive mode. In some embodiments, said controller is further configured to provide an alert when one or more of the measurements is outside a predetermined range. In some embodiments, said controller is configured to enter a turbo mode (a mode wherein the apparatus makes a greater number of measurements to more closely monitor the out of range measurement) to measure the liquid-quality parameters at more frequent intervals. In some embodiments, said controller is further configured to disconnect power to the apparatus if said alert is indicative of a water flow value at or below a predetermined value. In some embodiments, said controller is further configured to connect said at least one sensor after a predetermined period of time.

In some embodiments, said at least one sensor comprises a turbidity detector configured to detect illumination from said liquid sample at a 90-degree angle with respect to an illumination beam generated by an illuminator and impinging on said liquid sample, thereby measuring a turbidity thereof.

In some embodiments, the apparatus is further configured to determine a temperature compensation for the turbidity measurement using an illumination detector disposed at a 180 degree angle to the illumination beam in order to measure the illumination beam.

In some embodiments, the apparatus is configured to first perform a turbidity measurement, prior to any other measurements. In some embodiments, the turbidity sensor uses a colorimeter and the chlorine sensor does not use a colorimeter.

In some embodiments, the apparatus is configured to measure/analyze any one or combination of chlorine concentration; turbidity; and color. The apparatus typically also is configured to analyze the aforementioned measurements.

The terms “liquid” and “water” may be used interchangeably herein the specification and claims to refer to any liquid suitable for measurement and analysis by the present apparatus and method.

In accordance with embodiments of another aspect of the present invention there is provided a method of low energy chlorine and/or turbidity and/or color measurement of a liquid. The method includes: (a) retaining said sample of water from a water flow; (b) analyzing said water-quality parameter using at least one sensor selected from the group containing: a chlorine sensor; a turbidity sensor; a pH sensor; a temperature sensor; a pressure sensor; and a redox sensor, of a water quality measurement apparatus; and (c) controlling the operation of said apparatus between an active mode, when the apparatus is performing measurements; and a sleep mode when the apparatus is in a non-measurement, minimally powered state, wherein said controlling comprises operating an energy source management module, operably associated with said controller, to manage voltage in said controller and provide for extended power and low electricity consumption.

In some embodiments, step (c) further comprises controlling operation of the apparatus between said active mode, said sleep mode and a turbo-mode, which is employed in the event that measurement of the water-quality parameter is outside a given range.

In some embodiments, step (b) comprises the chlorine sensor measuring free chlorine or total chlorine. In some embodiments, step (b) comprises measuring a plurality of water-quality parameters a single liquid sample.

In some embodiments, step (c) further comprises the controller maintaining low power to said at least one sensor so that the sensor does not enter a passive mode. In some embodiments, step (c) further comprises the controller providing an alert when a measurement is outside a predetermined range. In some embodiments, step (c) further comprises the controller disconnecting said at least one sensor if said alert is indicative of a water flow value at or below a predetermined value. In some embodiments, step (c) further comprises the controller connecting said at least one sensor after a predetermined period of time.

In some embodiments, step (b) comprises detecting illumination from said liquid sample at a 90-degree angle with respect to an illumination beam generated by an illuminator and impinging on said liquid sample, thereby measuring turbidity thereof. In some embodiments, step (b) comprises first performing a turbidity measurement, prior to any other measurements. In some embodiments, step (b) further includes compensating for the temperature during the turbidity measurement using an illumination detector disposed at a 180-degree angle to the illumination beam; and/or determining a temperature compensation using an illumination detector disposed at a 180-degree angle to the illumination beam in order to measure the illumination beam.

In some embodiments of the present apparatus (analyzer), the chlorine measurement is made via a dedicated sensor electrode, rather than via a colorimeter of the apparatus. The colorimeter only tests turbidity. Additionally, measurements are not performed simultaneously, rather sequentially one after the other. There is one line for turbidity measurement and another for chlorine and other measurements. Turbidity is tested with a RGB sensor with color and chlorine is measured with a chlorine electrode/sensor, as noted above.

With one sample, the sampling cell can measure several parameters, such as chlorine (free chlorine and total chlorine), pH, redox, temperature and flowrate. Such multi-parameter measurements from a single sample obviate the need to retrieve several samples of the liquid and analyze them separately.

For the turbidity measurement, a colorimeter may be used, and since a colorimeter may not be required for chlorine measurement, chlorine may be measured with its own dedicated electrode/sensor. In preferred embodiments, there is first a turbidity measurement of a fresh water sample, and subsequently other measurements. Such protocol saves energy in colorimeter testing. The controller has an algorithm to ensure that the proper quantity of water enters, at right time, to make the measurement over the necessary time duration.

It is a particular feature of some embodiments of the present invention that the liquid/water quality measurement apparatus and method is configured to manage the voltage in the controller in order to ensure low electricity consumption.

It is also a particular feature of some embodiments of the present invention that the apparatus includes a battery/energy source management module, enabling provision of extended power, for example, three years of power instead of merely 1.5 years. In some embodiments the battery/energy source management module includes an analyzer configured to work with and operate the voltage in the apparatus efficiently. Although a typical battery of such apparatus has a lifetime of about four to six months, the present operation method manages operation of the apparatus such that the battery is used only when the apparatus is “awake” and thus the battery can last up to three years. The apparatus operation program is designed to minimize the active operational time of the apparatus, while using components that are designed to work in a low power environment. Specifically, the program/apparatus is designed to work in several states while operating, for example, including turbidity measuring, conductivity measuring, and/or measurement of other parameters. Additionally, following measurement of the water sample(s), the measurement results are transferred to the modem for communication and transmission to the server.

It is another particular feature of some embodiments of the present invention that the liquid/water quality measurement apparatus and method are configured so that between testing cycles (water sampling), the controller goes into a sleep mode, and at that time of sleep mode the analyzer is programmed to maintain low power on the electrodes/sensors so they do not enter a passive mode. For example, while the analyzer is in sleep mode, the circuit maintains very low voltage, just enough to keep the chlorine electrode from entering a passive state. The minimal energy required for this functionality may be drawn from the batteries (e.g. a set of twelve batteries), that are typically sufficient to provide up to about 3 years of power at the aforementioned level of functionality.

It is another particular feature of some embodiments of the present invention that total chlorine as well as free chlorine can be measured in a single sample.

In addition, the electronic components of the analyzer cards are selected to work in a low power environment, so that the electrodes will not go into passivation state (e.g. the controller keeps the chlorine electrode minimally “awake” (powered) to prevent the need for recalibration and thus the apparatus is ready to perform a subsequent chlorine level measurement when necessary/desired; after a turbidity measurement.

### BRIEF DESCRIPTION OF THE DRAWINGS

The principles and operation of the apparatus and method according to the present invention may be better understood with reference to the drawings, and the following description, it being understood that these drawings are for illustrative purposes only and are not meant to be limiting, wherein:

FIG. 1 is a schematic depiction of an apparatus for monitoring water quality, in accordance with embodiments of the present invention;

FIG. 2 is a flow diagram illustrating a method of analyzing water quality in accordance with embodiments of the present invention;

FIG. 3 is an illustration of a turbidity and chlorine content (CTC) analysis apparatus in accordance with a embodiments of the present invention;

FIG. 4 is an exploded view of a CTC measurement module of the apparatus of FIG. 3;

FIG. 5 is an illustration of an illumination and detection assembly, forming part of the CTC measurement module;

FIGS. 6A and 6B are simplified pictorial side views of a base element forming part of the illumination and detection assembly;

FIGS. 7A and 7B are illustrations of a detector assembly forming part of the illumination and detection assembly of FIG. 3; and

FIGS. 8A-8F are flowcharts illustrating a mode of operation of the apparatus, in accordance with embodiments of the present invention.

It will be appreciated that for simplicity and clarity of illustration, elements shown in the drawings have not necessarily been drawn to scale. For example, the dimensions of some of the elements may be exaggerated relative to other elements for clarity. Further, where considered appropriate, reference numerals may be repeated among the drawings to indicate corresponding or analogous elements throughout the figures.

### DETAILED DESCRIPTION OF EMBODIMENTS OF THE INVENTION

The following description is presented to enable one of ordinary skill in the art to make and use the invention as provided in the context of a particular application and its requirements. Various modifications to the described embodiments will be apparent to those with skill in the art, and the general principles defined herein may be applied to other embodiments. Therefore, the present invention is not intended to be limited to the particular embodiments shown and described, but is to be accorded the widest scope consistent with the principles and novel features herein disclosed. In other instances, well-known methods, procedures, and components have not been described in detail so as not to obscure the present invention.

Embodiments of the invention enable low energy liquid (e.g. water) measurement/analysis, for example, of chlorine content or concentration, turbidity, pH, temperature, pressure and conductivity. Some embodiments provide an apparatus and method for simultaneous or near simultaneous measurement of the turbidity and/or chlorine content of a sample of a liquid.

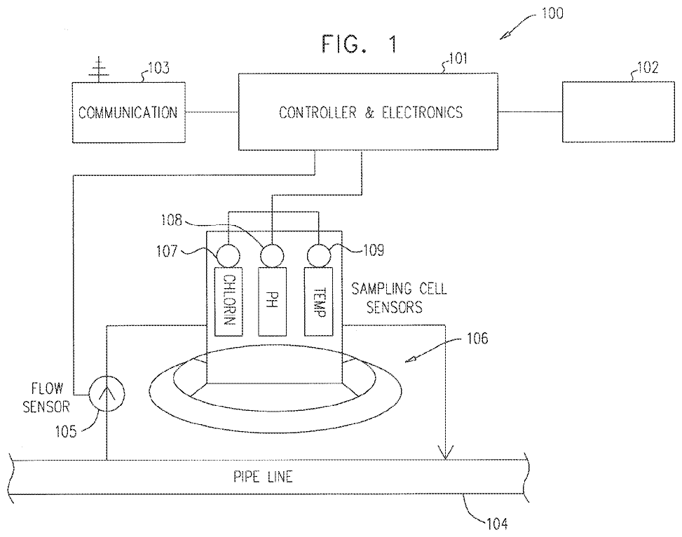

FIG. 1 shows a schematic of an embodiment of an apparatus 100 for monitoring water quality. Apparatus 100 is configured to measure pH, temperature, and chlorine concentration in water in a pipe line 104, and is further configured to analyze the measurements; to store data associated with the measurements, which may include the measurements and results of performed analyses; and to output the data through a local interface and/or remote interface. Apparatus 100 includes a sampling cell 106; a chlorine sensor 107, having a chlorine-sensing electrode (not shown); a pH sensor 108; a water temperature sensor 109; a flow sensor 105; a controller 101 including associated electronic circuitry and peripherals; a communications module 103; and a power module 102.

Monitoring water quality using apparatus 100 is typically performed by diverting a portion of the water in pipe line 104 into sampling cell 106, which includes chlorine sensor 107, pH sensor 108 and water temperature sensor 109. Chlorine sensor 107, pH sensor 108, and water temperature sensor 109 are configured to perform water quality measurements of the water flowing through sampling cell 106, and may be any suitable commercially available sensors. Optionally, chlorine sensor 107, pH sensor 108, and water temperature sensor 109 are configured to perform water quality measurements of the water flowing through pipe line 104. Flow sensor 105 is configured to measure the water flow rate into sampling cell 106 and, optionally, in pipe line 104.

Controller 101 includes peripherals and associated control circuitry required for operating apparatus 100, including controlling the operation of communications module 103, power module 102, and all the sensors. Controller 101 is configured to receive measurement inputs from flow sensor 105, chlorine sensor 107, pH sensor 108, and water temperature sensor 109; as well as readings of conductivity, pressure, redox and turbidity, and to process the measurements and to analyze the quality of the water. Controller 101 is further configured to control apparatus 100 to be in an active mode of operation, a sleep mode or a shut-down mode, responsive to the inputs received from the sensors; and/or responsive to external signals from sources external to apparatus 100; and/or responsive to periodic time initiations and/or non-periodic time initiations. In some embodiments, the apparatus is configured to operate in a mode termed “turbo mode”, which the apparatus enters when the value of the results is out of a defined range. Turbo mode is a mode wherein controller 101 instructs the apparatus to take relatively frequent measurements so as to more closely monitor such “out of range value” situations. External signals from sources external to the apparatus may be referred to herein as external interrupts, and periodic and non-periodic time initiations may be referred to as time interrupts. Controller 101 optionally is adapted to perform a self-test to evaluate proper operation of some, or optionally all, functions of apparatus 100.

Communications module 103 is adapted to enable communications between apparatus 100 and other communication devices physically located in close proximity (local interfacing) and/or distantly located (remote interfacing). Interfacing may be performed while apparatus 100 is in the active mode.

Local interfacing between apparatus 100 and external devices such as, for example, external controllers and/or storage mediums, may be done by means of a USB connection and/or other type of wired data transfer connection. Optionally, local interfacing is done using removable storage means such as flashcards, and the like. Optionally, local interfacing is done using wireless means such as, for example, a WLAN (wireless local area network). The WLAN may conform to IEEE standards 802.11 (Wireless LAN-WiFi), and/or IEEE Standards 802.15 (Wireless PAN-WPAN).

Remote interfacing between apparatus 100 and other communication devices is generally through wireless means. Communications module 103 is configured to remotely interface via GRRS.GSM communications, which may include direct antenna to antenna microwave links, satellite communications, cellular phone networks, and/or through a WLAN. The WLAN may conform to IEEE standard 802.16 (Broadband Wireless Access-WiMAX), 802.20 (Mobile Broadband Wireless Access-MBWA), and/or 802.22 (Wireless Regional Area Network-WRAN), or any combination thereof. Optionally, remote interfacing is through wire communications means such as, for example, dedicated cables, and/or power lines.

Communications module 103 is configured to transmit data associated with the aforementioned measurements, which may include the measurement and analysis results. Optionally, data transmitted may include data related to the operational status of the apparatus, and warnings/alarms related to equipment malfunction and/or to poor water quality. Communications module 103 may be further configured to receive external interrupts, and optionally, prompts or requests for data. Optionally, communications module 103 may be configured to receive and transfer to controller 101 reprogramming instructions/information.

Power module 102 includes a battery package configured to serve as a DC voltage source for powering apparatus 100. Power module 102 may optionally include an AC/DC voltage converter for connection of the apparatus to power lines. Additionally or alternatively, power module 102 may be connected to a generator. Optionally, power module 102 may be connected through a USB interface for power supply from a PC, laptop computer, or other USB interface DC power supply source. It is a particular feature of some embodiments of the present invention that power module 102 is configured and/or managed to provide extended power, for example, three years of power instead of merely about half a year.

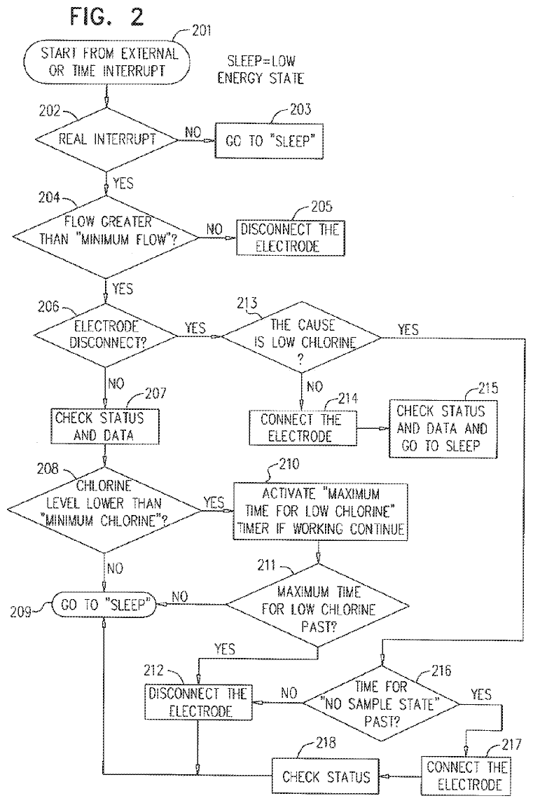

FIG. 2 shows a flow diagram of an algorithm for implementing a method for using apparatus 100 to measure chlorine concentration, in accordance with embodiments of the invention. It may be appreciated by a person skilled in the art that the algorithm described below is for illustrative purposes; that there may be numerous other steps that may be implemented in the algorithm, and that the algorithm described below is in not intended to be limiting.

[STEP 201] An interrupt signal is received by controller 101 while apparatus 100 is in sleep mode or shut-down mode. The interrupt signal may be either an external interrupt received through a local interface or a remote interface. Optionally, the interrupt signal may be predetermined and periodic, or alternatively, non-periodic.

[STEP 202] Controller 101 verifies if the interrupt signal is an external or internal interrupt signal. If the signal is not an external or an internal interrupt signal, go to STEP 203. If the signal is either an external or an internal interrupt signal, go to STEP 204.

[STEP 203] Apparatus 100 goes into sleep mode. In the sleep mode, functions in apparatus 100 can be disconnected to further reduce power consumption in addition to the functions of in chlorine sensor 107. Chlorine sensor 107 (including the electrode thereof) is energized. It is a particular feature of some embodiments of the present invention that the liquid/water quality measurement apparatus and method are configured so that between testing cycles (water sampling), controller 101 goes into a sleep mode, and at that time (sleep mode) the analyzer is programmed to maintain low power on the electrodes/sensors so they do not enter a passive mode. As such, the apparatus is typically ready with no or limited delay to perform one or more water-quality measurements/analyses.

[STEP 204] Controller 101 processes measurement input from flow sensor 105 to determine if the water flow rate is greater than a predetermined minimum value. If the water flow rate is less than or equal to the predetermined minimum value, go to STEP 205. If the water flow rate is greater than the predetermined minimum value, go to STEP 206.

[STEP 205] Apparatus 100 goes into a shut-down mode. Power to the electrode in chlorine sensor 107 is disconnected, as well as to most other functions in the chlorine sensor. In the shut-down mode, functions in apparatus 100 may optionally be disconnected to further reduce power consumption of apparatus 100, in addition disconnecting chlorine sensor 107.

[STEP 206] Controller 101 checks if the electrode in chlorine sensor 107 is disconnected. If electrode is not disconnected, go to STEP 207. If electrode is disconnected, go to STEP 213.

[STEP 207] Controller 101 receives and processes measurement data from chlorine sensor 107.

[STEP 208] Controller 101 compares measured chlorine concentration in the water with a predetermined minimum value. If the measured chlorine concentration is equal to or greater than a predetermined minimum value, go to STEP 209. If the measured chlorine concentration is less than the predetermined minimum value, go to STEP 210.

[STEP 209] Apparatus 100 goes into sleep mode.

[STEP 210] Controller 101 periodically compares, typically at a predetermined time interval, the measured chlorine concentrations in the water with the predetermined minimum value.

[STEP 211] If the measured chlorine concentration is equal to or greater than the predetermined minimum value during the predetermined time interval, go to STEP 209. If the measured chlorine concentration is less than the predetermined minimum value during the predetermined time interval, go to STEP 212.

[STEP 212] Apparatus 100 goes into shut-down mode; the power to chlorine sensor 107 is disconnected.

[STEP 213] Controller 101 checks if the chlorine sensor's electrode is disconnected because of previously measured low chlorine concentrations in the water. If not disconnected because of previously measured low chlorine concentrations in the water, go to STEP 214. If the chlorine sensor's electrode is disconnected because of previously measured low chlorine concentrations in the water, go to STEP 216.

[STEP 214] Controller 101 activates chlorine sensor 107 and energizes the chlorine sensor's electrode.

[STEP 215] Controller 101 receives and processes measurement data from chlorine sensor 107; apparatus 100 goes into sleep mode.

[STEP 216] Controller 101 checks if the time passed since the last measurement is greater than a predetermined time interval. If the time passed is less than the predetermined time interval, go to STEP 212. If the time passed is greater than or equal to the predetermined time interval, go to STEP 217.

[STEP 217] Controller 101 activates chlorine sensor 107 and energizes the electrode.

[STEP 218] Controller 101 receives and processes measurement data from chlorine sensor 107. Go to STEP 109.

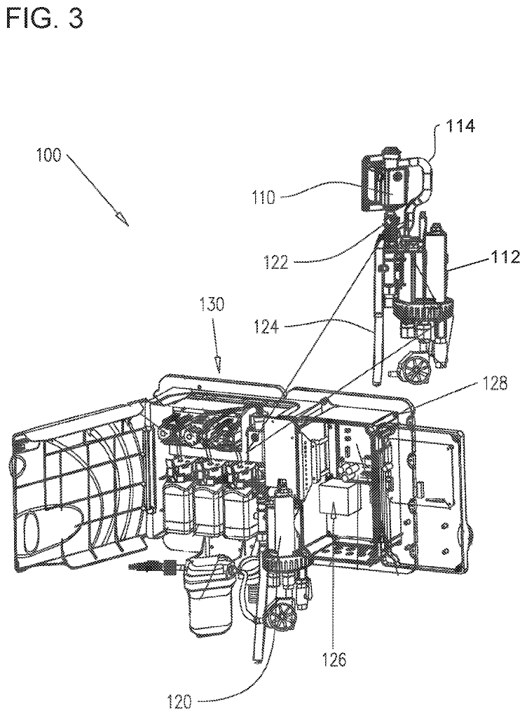

FIG. 3 shows apparatus 100 configured as a turbidity and chlorine content (CTC) measurement/analysis apparatus in accordance with embodiments of the present invention. Apparatus 100 includes a colorimeter 112 having a colorimeter water outlet 114. Colorimeter 112 is designed to measure turbidity only, whereas the chlorine measurements are performed using a separate and dedicated chlorine electrode with chlorine sensor 107. Apparatus 100 is operable for rapid successive measurement of turbidity and chlorine by: (a) retaining, from a continuous flow of the liquid, a sample volume of the liquid; and (b) detecting illumination from the sample volume. This detecting from the sample volume can include: (i) detecting by a first detector operable for detecting illumination from the sample volume of liquid at a 90-degree angle with respect to an illumination beam generated by an illuminator and impinging on a sample volume of the liquid, thereby measuring a turbidity of the sample volume of liquid; and/or (ii) detecting by a second detector configured to detect illumination from the sample volume of liquid at a 180-degree angle with respect to the illumination beam, thereby measuring a color of the sample volume of liquid.

CTC measurement module 110 is configured to receive samples of liquid to be analyzed from a sampling cell assembly 120, via a solenoid valve 122. CTC measurement module 110 is also configured to output liquid contained therein, such as analyzed samples of liquid or liquid used for cleaning the interior of the CTC measurement module, via a drain pipe 124. Sampling cell assembly 120 (e.g. Blue-I Water Technologies Ltd., Rosh Ha'ayin, Israel, Catalog No. 970-210-3120).

The operation of CTC measurement module 110 is controlled by a computerized controller assembly 126, which is typically enclosed in a protective enclosure 128. Enclosure 128 is typically separate from and adjacent to an enclosure 130, which houses CTC measurement module 110 together with part of sampling cell assembly 120. In addition to the specific operation of CTC measurement module 110 described hereinbelow, parts of the structure and operation of apparatus 100 are described in U.S. Pat. No. 7,662,342 of the Applicant, the disclosure of which is hereby incorporated by reference.

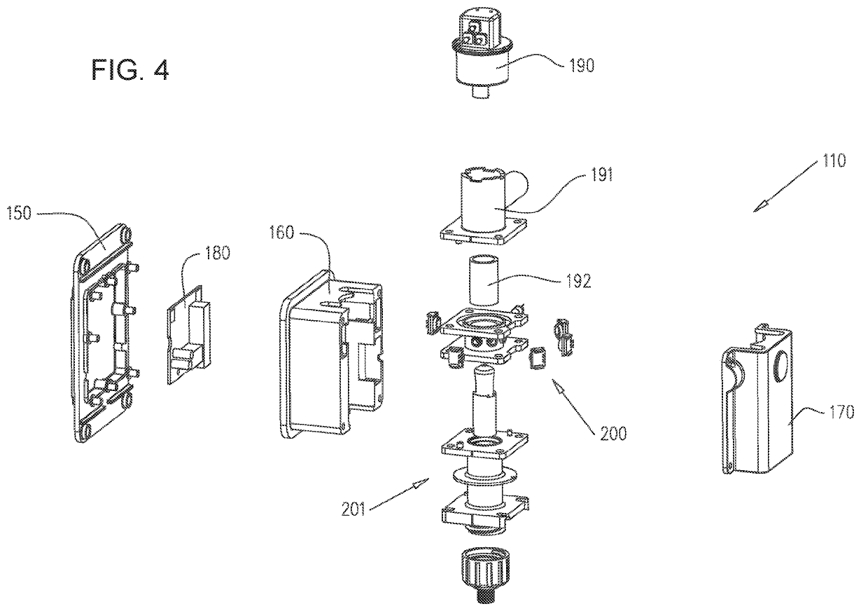

FIG. 4 shows an exploded view of CTC measurement module 110. In some embodiments, CTC measurement module 110 includes a base element 150 (e.g. Blue-I Water Technologies Ltd., Rosh Ha'ayin, Israel, Catalog No. 1-COVER-PCB). A housing element 160 is mounted onto base element 150. Housing element 160 (e.g. Blue-I Water Technologies Ltd. of Rosh Ha'ayin, Israel, Catalog No. 970-210-3004). Also mounted onto base element 150 is a light-tight housing element cover 170.

A calibration memory board 180 is disposed within a housing defined by base element 150; housing element 160; and housing element cover 170. Calibration memory board 180 includes a suitably programmed EPROM (e.g. I2C serial EEPROM), Microchip Technology of Chandler, Ariz., USA Catalog No. 24AA08/24LC08B) or the like.

A colorimeter head 190 (e.g. Blue-I Water Technologies Ltd. of Rosh Ha'ayin, Israel, Catalog No. 970-210-3018 or Catalog No. 970-210-3019) is also disposed within the housing defined by base element 150, housing element 160 and housing element cover 170. Colorimeter head 190 is supported by a measuring head 191, such as a measuring head commercially available from Blue-I Water Technologies Ltd. of Rosh Ha'ayin, Israel, Catalog No. 970-210-3014.

Colorimeter head 190 is designed to transfer water into a liquid sample, which is held in a transparent glass sample holder 192, such as a glass sample holder commercially available from Blue-I Water Technologies Ltd. of Rosh Ha'ayin, Israel, under Catalog No. 970-210-3017.

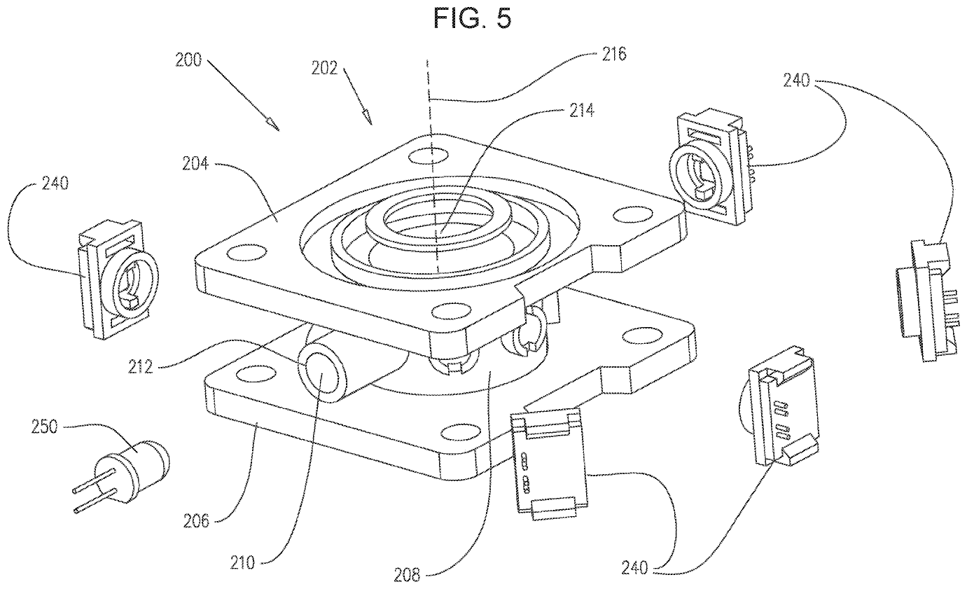

An illumination and detection assembly 200 is arranged to support sample holder 192 and to be in optical communication therewith, as described hereinbelow in detail with reference to FIGS. 5-7B.

In some embodiments, associated with sample holder 192 is a sample holder cleaning assembly 201 (e.g. Blue-I Water Technologies Ltd. of Rosh Ha'ayin, Israel, Catalog Nos. 970-210-3101 and 970-210-3204).

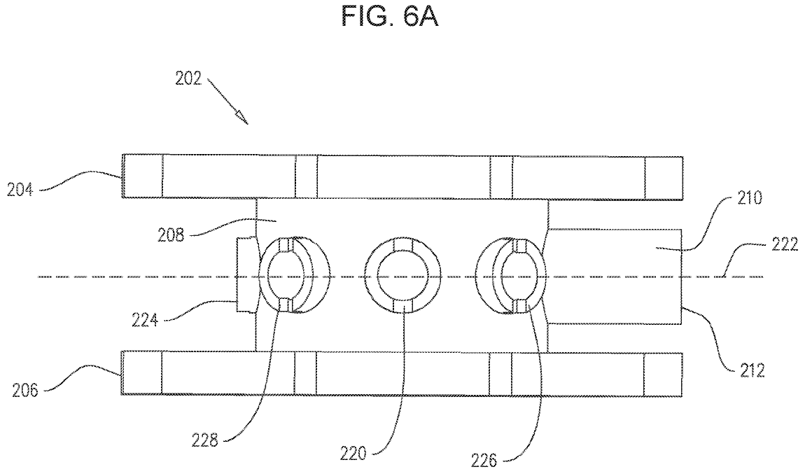

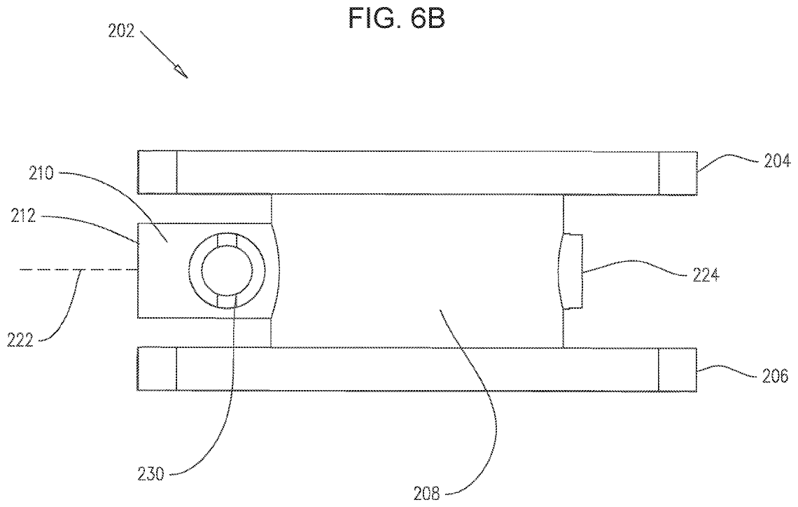

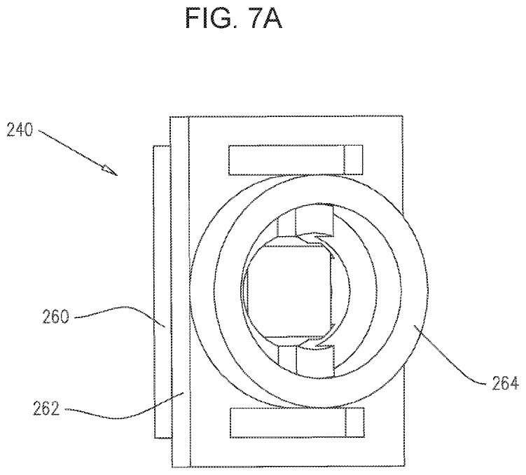

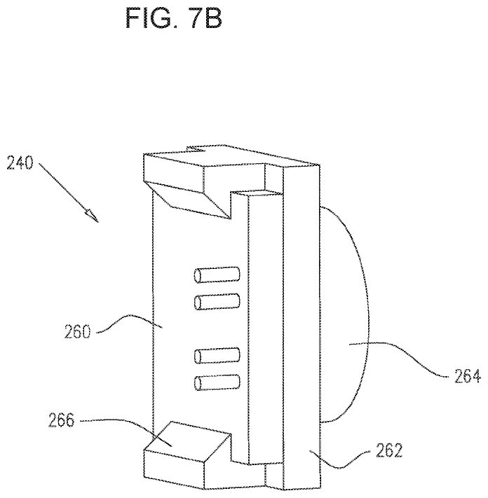

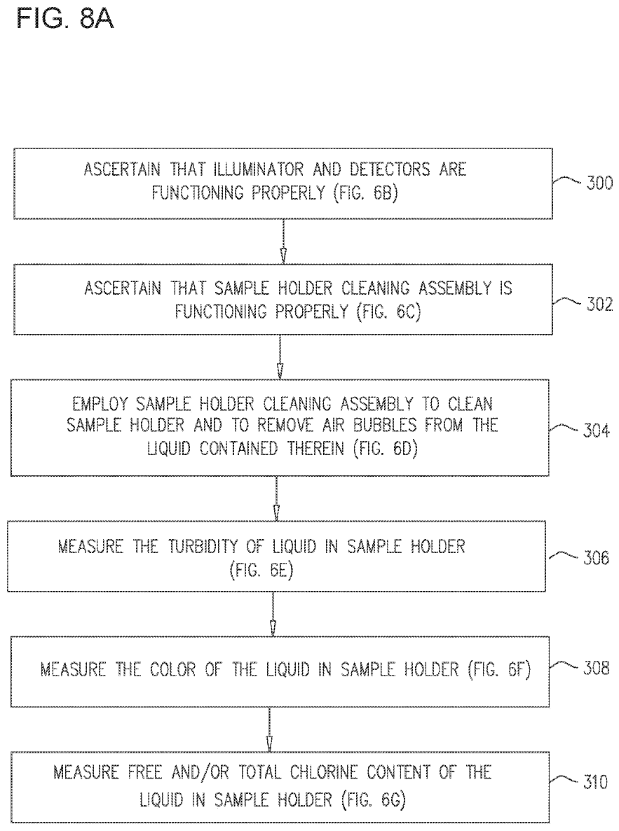

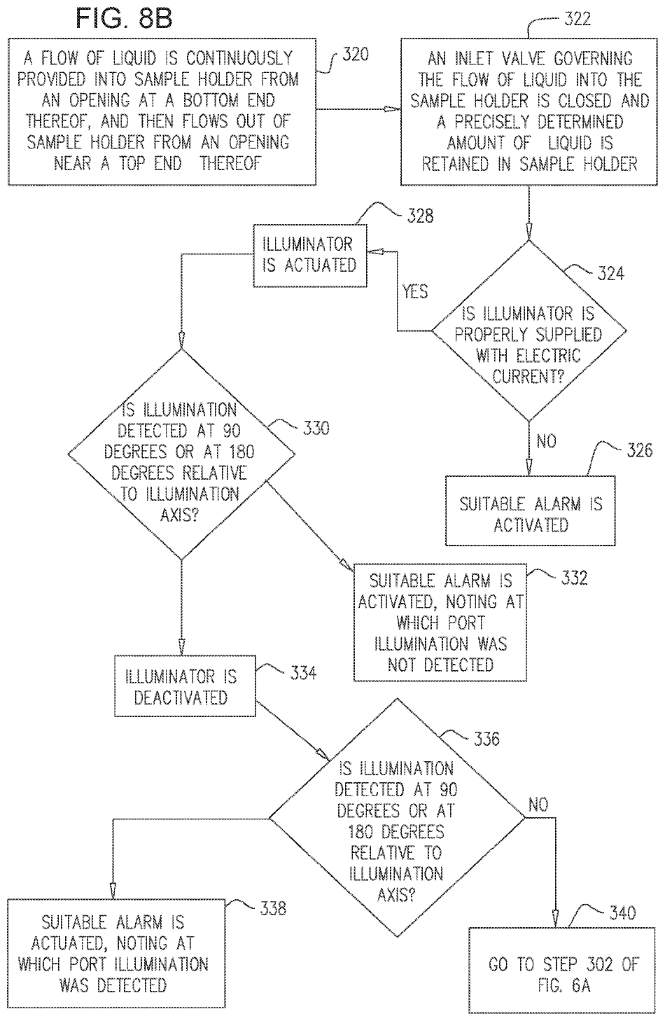

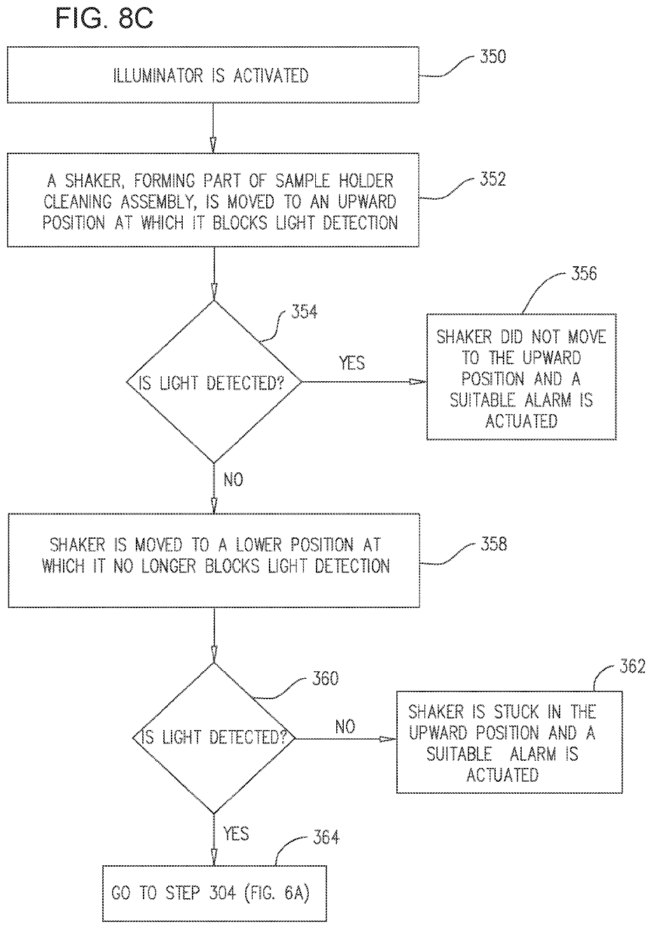

FIG. 5 shows a simplified exploded view of illumination and detection assembly 200, and FIGS. 6A and 6B show simplified opposing side views of a base element 202 thereof. Illumination and detection assembly 200 includes a base element 202, formable by plastic injection molding. Base element 202 includes respective top and bottom plate portions 204 and 206, which are joined by a generally cylindrical portion 208. An illumination conduit 210 intersects cylindrical portion 208. An illuminator port 212 is formed at an end of illumination conduit 210.

A bore 214 is formed through top plate portion 204, generally cylindrical portion 208 and bottom plate portion 206 of base element 202, along an axis 216, which is generally perpendicular to a top surface of top plate portion 204. Bore 214 is configured to receive sample holder 192.

As seen in FIG. 6A, generally cylindrical portion 208 is formed with multiple detector mounting ports arranged for light-tight mounting of light detector assemblies thereon, for turbidity measurements. The detector mounting ports include a first detector mounting port 220 located perpendicular to an illumination axis 222 defined by illumination conduit 210, and a second detector mounting port 224 located opposite illuminator port 212 along illumination axis 222. Additional optional detector mounting ports 226 and 228 are respectively arranged at 45 and 150 degree angles relative to illumination axis 222.

As seen in FIG. 6B, an illumination test detector port 230 is provided on illumination conduit 210, perpendicular to illumination axis 222.

Detector assemblies 240 (FIGS. 5, 7A and 7B) are removably mounted onto each of detector mounting ports 220, 224, 226, 228 and 230 in a light-tight manner. An LED illuminator 250, such as a YZ-W5S20N LED lamp (e.g. from YolDal Ltd. of Zhonghe City Taiwan), can be removably mounted onto illuminator port 212 of illumination conduit 210. Illuminator 250 is configured to illuminate an interior volume of bore 214, thereby illuminating liquid contained within transparent glass sample holder 192. Detector assemblies 240 are operable for detecting illumination generated by illuminator 250 and which traverses liquid contained within transparent glass sample holder 192.

FIGS. 7A and 7B are simplified pictorial illustrations of detector assembly 240 forming part of illumination and detection assembly 200 of FIG. 5. Detector assembly 240 includes a detector 260 (e.g. Texas Advanced Optoelectronic Solutions Inc., Piano, Tex., catalog number TCS 3403 or TCS 3413), and a detector mount 262. Detector mount 262 includes a port connector portion 264, which is configured for tight engagement with any of ports 220, 224, 226, 228 and 230 in a light-tight manner. Detector mount 262 also includes a detector mounting portion 266, which is configured to retain detector 260 to port connector portion 264 in a light-tight manner.

Detectors 260 are operative both as an ambient light sensor and an RGB color sensor. Additionally or alternatively, detectors 260 may be operative to detect a specific wavelength, or may be fitted with a filter operative to filter only a specific wavelength.

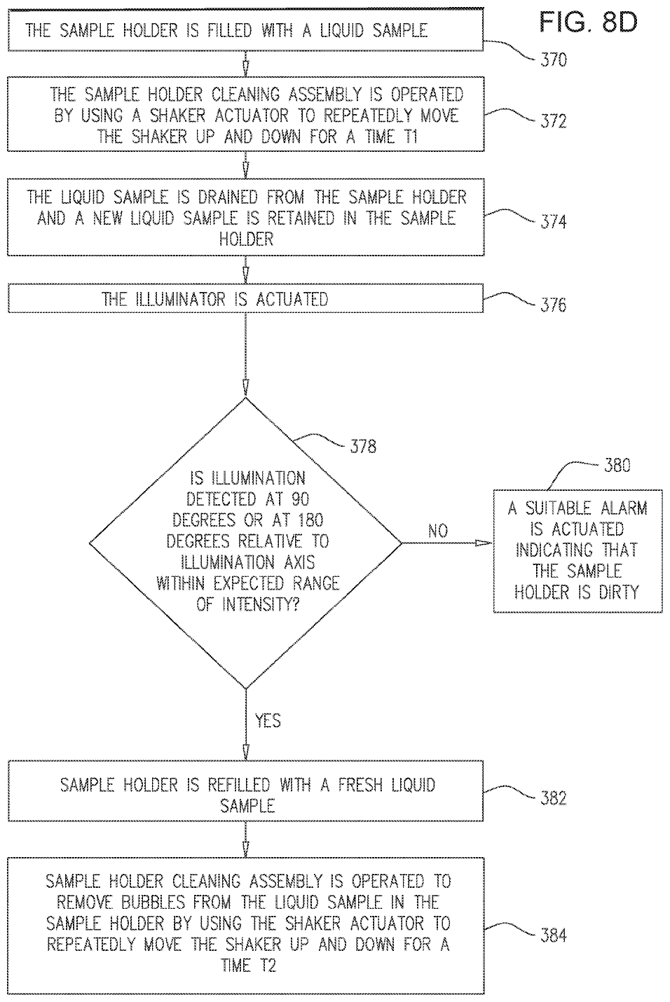

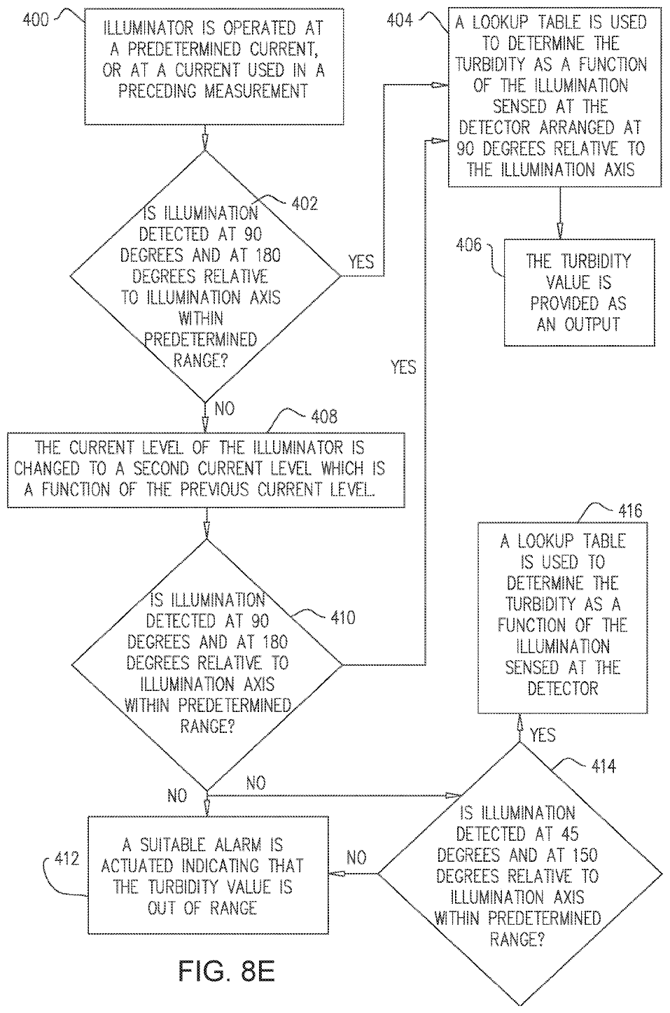

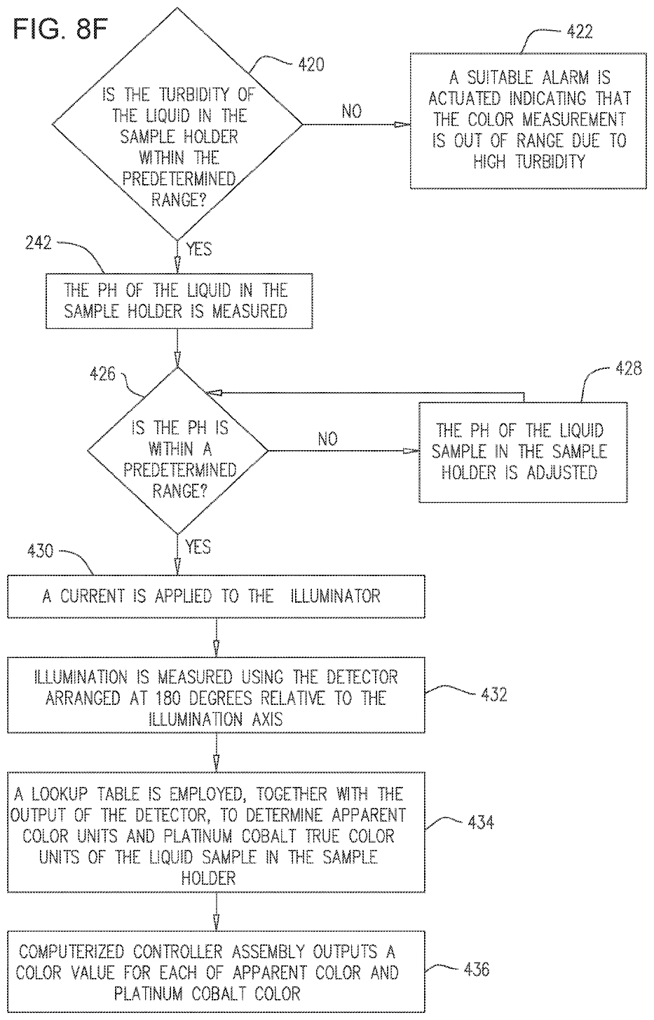

FIGS. 8A-8G show embodiments of an operation mode of apparatus 100 shown in FIGS. 3-7B. As seen in FIG. 8A, the operation of apparatus 100 includes the following principal steps:

ascertaining that illuminator 250 and detector assemblies 240 are functioning properly, as will be described in detail hereinbelow with reference to FIG. 8B (step 300); ascertaining that sample holder cleaning assembly 201 is functioning properly, as will be described in detail hereinbelow with reference to FIG. 8C (step 302);

employing sample holder cleaning assembly 201 to clean sample holder 192 and to remove air bubbles from the liquid contained therein, as will be described in detail hereinbelow with reference to FIG. 8D (step 304);

measuring the turbidity of liquid in sample holder 192, as will be described in detail hereinbelow with reference to FIG. 8E (step 306);

measuring the color of the liquid in sample holder 192, the turbidity of which was measured in step 306, as will be described in detail hereinbelow with reference to FIG. 8F (step 308); and/or measuring free and/or total chlorine content of the liquid in sample holder 192 via the electrode of chlorine sensor 107, the turbidity of which was measured in step 306, as will be described in detail hereinbelow with reference to FIG. 8G (step 310).

FIG. 8B shows step 300 (FIG. 8A), which includes ascertaining that illuminator 250 and detector assemblies 240 are functioning properly.

As shown in step 320 of FIG. 8B, a flow of liquid is generally continuously provided into sample holder 192 from an opening at a bottom end thereof, and then flows out of sample holder 192 from an opening near a top end thereof. As further shown in step 322, intermittently, and typically periodically, an inlet valve governing the flow of liquid into the sample holder 192 is closed and a precise amount of liquid is retained in sample holder 192. The liquid is typically drinking water, however the liquid may be any other liquid for which measuring of any of turbidity, color and chlorine content is desired.

In step 324, apparatus 100 ascertains that illuminator 250 is properly supplied with electric current, or else a suitable alarm is activated (step 326). Responsive to ascertaining that illuminator 250 is properly supplied with electric current, illuminator 250 is actuated (step 328) and the outputs of detectors 260 mounted on ports 220 and 224, arranged at 90 degrees and 180 degrees respectively relative to illumination axis 222, are received and analyzed to ascertain whether illumination has been detected (step 330). Failure to detect illumination at either one of detectors 260 mounted on ports 220 and 224 causes a suitable alarm to be activated, noting at which of ports 220 and 224 illumination was not detected (step 332).

Alternatively or additionally, the output of detector 260 at port 230 is also received and analyzed. Failure to detect illumination at this detector also causes a suitable alarm to be activated.

If detectors 260 mounted on both ports 220 and 224 detect illumination, illuminator 250 is deactivated (step 334) and the outputs of detectors 260 at ports 220 and 224 are again received and analyzed to ascertain whether illumination has been detected, thereby ascertaining light tightness of the illumination and detection assembly of FIG. 5 (step 336). If light is detected, a suitable alarm is actuated, noting at which of ports 220 and 224 illumination was detected (step 338). If no light is detected, the process continues with step 302 of FIG. 8A (step 340).

FIG. 8C shows step 302 (FIG. 8A), which includes ascertaining that sample holder cleaning assembly 201 is functioning properly.

FIG. 8C shows that illuminator 250 is initially activated (step 350). While illuminator 250 is activated, a shaker, forming part of sample holder cleaning assembly 201, is moved to an upward position so as to block light detection by detector 260 at port 224 (step 352). Detection of light at this stage by detector 260 at port 224 (step 354) is an indication that the shaker did not move to the upward position and a suitable alarm is actuated (step 356).

If no light is detected at this stage by detector 260 at port 224, the shaker is then moved to a lower position wherein the shaker no longer blocks light detection by detector 260 at port 224 (step 358). No detection of light at this stage by detector 260 at port 224 (step 360) is an indication that the shaker is stuck in the upward position and a suitable alarm is actuated (step 362). If light is detected at this stage by detector 260 at port 224, the process continues with step 304 of FIG. 8A (step 364).

FIG. 8D shows step 304 (FIG. 8A), which includes employing sample holder cleaning assembly 201 to clean sample holder 192 and to remove air bubbles from the liquid contained therein.

As shown in FIG. 8D, once sample holder 192 is filled with a liquid sample (step 370), sample holder cleaning assembly 201 is operated by using a shaker actuator to repeatedly move the shaker up and down for a time T (step 372). The liquid sample is then drained from the sample holder and a new liquid sample is retained in the sample holder (step 374).

Thereafter, illuminator 250 is actuated (step 376) and the outputs of detectors 260 mounted on ports 220 and 224, arranged at 90 degrees and 180 degrees respectively relative to illumination axis 222, are received and analyzed to ascertain whether illumination has been detected (step 378). Failure to detect illumination at either of detectors 260 mounted on ports 220 and 224, or detection of illumination at either of detectors 260 mounted on ports 220 and 224 that is outside an expected range of intensity, a suitable alarm is actuated indicating that the sample holder 192 is dirty (step 380). If illumination detected at both detectors 260 mounted on ports 220 and 224 is within the expected range of intensity, sample holder 192 is refilled with a fresh liquid sample (step 382) and sample holder cleaning assembly 201 is operated to remove bubbles from the liquid sample in the sample holder 192 by using the shaker actuator to repeatedly move the shaker up and down for a time T2 (step 384).

FIG. 8E shows step 306 (FIG. 8A), which includes measuring the turbidity of liquid in sample holder 192.

To measure the turbidity of the liquid in sample holder 192, the illuminator 250 is initially operated at a predetermined current, or at a current used in a preceding measurement (step 400). The outputs of detectors 260 mounted on ports 220 and 224 arranged at 90 degrees and 180 degrees respectively relative to illumination axis 222 are received and analyzed to ascertain whether the illumination detected at detectors 260 mounted on ports 220 and 224 is within a predetermined range of intensity (step 402).

Responsive to ascertaining that the intensity of the illumination detected at detectors 260 at ports 220 and 224 is within a predetermined range of intensity, a lookup table is used to determine the turbidity as a function of the intensity of the illumination detected at detector 260 mounted on port 220, arranged at 90 degrees relative to illumination axis 222 (step 404), and the turbidity value is provided as an output (step 406). The lookup table can be based on a pre-calibrated light intensity/turbidity curve for detector 260 at port 220 arranged at 90 degrees relative to illumination axis 222. It is appreciated that the turbidity values are based on nephelometric analysis.

Responsive to ascertaining that the intensity of the illumination detected at detectors 260 at ports 220 and 224 is not within the predetermined range of intensity, the current level of illuminator 250 is changed to a second current level (step 408), which second current level is typically a function of the previous current level. Thereafter, the outputs of detectors 260 mounted on ports 220 and 224 arranged at 90 degrees and 180 degrees respectively relative to illumination axis 222 are again received and analyzed to ascertain whether the illumination detected at detectors 260 mounted on ports 220 and 224 are within the predetermined range of intensity (step 410). Responsive to ascertaining that the illumination detected at detectors 260 at ports 220 and 224 is within the predetermined range of intensity, a lookup table is used to determine the turbidity as a function of the intensity of the illumination detected at detector 260 mounted on port 220, arranged at 90 degrees relative to illumination axis 222 (step 404), and the turbidity value is provided as an output (step 406).

Responsive to ascertaining that the intensity of the illumination detected at detectors 260 mounted on ports 220 and 224 is still not within the predetermined range, a suitable alarm is actuated indicating that the turbidity value is out of range (step 412). Alternatively, the outputs of detectors 260 at port 226 and/or 228, arranged at 45 degrees and 150 degrees respectively relative to illumination axis 222, are received and analyzed to ascertain whether the illumination detected at detectors 260 mounted on port 226 and/or 228 is within a predetermined range (step 414). Responsive to ascertaining that the intensity of the illumination detected at detectors 260 mounted on ports 226 and/or 228 is within the predetermined range, a lookup table can be used to determine the turbidity as a function of the illumination detected at detector 260 mounted on port 226 or 228 (step 416). Responsive to ascertaining that the illumination detected at detectors 260 mounted on port 226 and/or port 228 are not within the predetermined range, a suitable alarm is actuated indicating that the turbidity value is out of range (step 412).

FIG. 8F shows step 308 (FIG. 8A), which includes measuring the color of the liquid in sample holder 192, the turbidity of which was measured in step 306. It is appreciated that the color of a liquid typically correlates with the level of contamination of the liquid. For example, drinking water may be colored as a result of contamination by material dissolved in the liquid such as, for example, soil or pipe corrosion.

Initially, the apparatus ascertains whether the turbidity of the liquid in sample holder 192 measured as described in FIG. 8E was within the predetermined range (step 420). Responsive to ascertaining that the turbidity was not within the predetermined range, a suitable alarm is actuated indicating that the color measurement is out of range due to high turbidity (step 422).

Responsive to ascertaining that the turbidity was within the predetermined range, the pH of the liquid in sample holder 192 is measured (step 424) and the apparatus ascertains whether the pH is within a predetermined range, typically a range of 4-10 (step 426). It is appreciated that the pH of the liquid may be measured before entering sample holder 192.

Responsive to ascertaining that the pH is not within the predetermined range, the pH of the liquid sample in sample holder 192 is adjusted (step 428). The adjustment of the pH is to within the predetermined range, typically to a value of 7.0 or to any other suitable pH, by adding an acid, base or buffer to the sample and by employing the shaker to mix the liquid sample in sample holder 192 while removing bubbles therefrom. Thereafter, a second pH measurement is performed on the same liquid sample in sample holder 192 to ascertain that the pH is within the predetermined range (step 426).

Responsive to ascertaining that the pH is within the predetermined range, a current is applied to illuminator 250 (step 430) and illumination is measured using the detector 260 at port 224, arranged at 180 degrees relative to illumination axis 222 (step 432). A lookup table can be employed, together with the output of detector 260 at port 224, to determine apparent color units and platinum cobalt true color units of the liquid sample in sample holder 192 (step 434).

The lookup table can include apparent color units (400-700 nm) and platinum cobalt true color units (450-465 nm) as a function of turbidity range (0-1000 ntu) and pH (4-10). The lookup table can be used to eliminate the influence of turbidity and pH on the detection and determination of color of the liquid sample. Based on the lookup table, computerized controller assembly 126 determines and outputs a color value for each of apparent color and platinum cobalt color (step 436).

FIG. 8G shows step 310 (FIG. 8A), which includes measuring free or total chlorine content of the liquid in sample holder 192, the turbidity of which was measured in step 306. The free chlorine content of a liquid typically correlates to the residual disinfecting power of the liquid, and that the total chlorine content of a liquid typically correlates to the overall level of contamination of the liquid.

The inlet valve is then reopened to allow fresh water to flow through sample holder 192 (step 458) and the shaker moves again to clean the colorimeter and prepare for the next reading (step 460).

The foregoing description of the embodiments of the invention has been presented for the purposes of illustration and understanding. It should be appreciated by persons skilled in the art that many modifications, variations, substitutions, changes, and equivalents are possible in light of the above description. It is, therefore, to be understood that the appended claims are intended to cover all such modifications and changes as fall within the scope of the invention.

1. An apparatus for measuring a water-quality parameter of a liquid sample, the apparatus comprising:
at least one water-quality parameter sensor selected from the group containing: a chlorine sensor; a turbidity sensor; a conductivity sensor; a pH sensor; a temperature sensor; a pressure; a redox sensor; and a flow sensor;
a controller configured to control operation of the apparatus between an active mode, when the apparatus is performing measurements; and a sleep mode when the apparatus is in a non-measurement, minimally powered state;
an energy source management module operably associated with said controller, wherein said module is configured to manage voltage in said controller and provide for extended power and low electricity consumption.

2. The apparatus of claim 1, wherein the controller is configured to further control operation of the apparatus between said active mode, said sleep mode, and a turbo-mode, which is a mode that is employed in the event that measurement of the water-quality parameter is outside a given range.

3. The apparatus of claim 1, wherein the chlorine sensor is configured to measure free chlorine or total chlorine.

4. The apparatus of claim 1, configured so that a plurality of water-quality parameter sensors of said at least one sensor are usable in a single liquid sample.

5. The apparatus of claim 1, wherein said controller is further configured to maintain low power to a Total Chlorine sensor and/or a Free Chlorine sensor so that the sensor remains in an active mode.

6. The apparatus of claim 1, wherein said controller is further configured to provide an alert when one or more of the measurements is outside a predetermined range.

7. The apparatus of claim 6, wherein said controller is configured to enter a turbo mode to measure the liquid-quality parameters at more frequent intervals.

8. The apparatus of claim 6, wherein said controller is further configured to disconnect power to the apparatus if said alert is indicative of a water flow value at or below a predetermined value.

9. The apparatus of claim 1, wherein said controller is further configured to connect said at least one sensor after a predetermined period of time.

10. The apparatus of claim 1, wherein said at least one sensor comprises a turbidity detector configured to detect illumination from said liquid sample at a 90-degree angle with respect to an illumination beam generated by an illuminator and impinging on said liquid sample, thereby measuring a turbidity thereof.

11. The apparatus of claim 10, wherein the apparatus is further configured to determine a temperature compensation using an illumination detector disposed at a 180-degree angle to the illumination beam in order to measure the illumination beam.

12. The apparatus of claim 1, being configured to first perform a turbidity measurement, prior to any other measurements.

13. The apparatus of claim 1, wherein the turbidity sensor uses a colorimeter and the chlorine sensor does not use a colorimeter.

14. A method of measuring a water-quality parameter of a liquid sample, the method comprising:
(a) retaining said sample of water from a water flow;
(b) analyzing said water-quality parameter using at least one sensor selected from the group containing: a chlorine sensor, a pH sensor, a temperature sensor, and a redox sensor, of a water quality measurement apparatus;
(c) controlling the operation of said apparatus between an active mode, when the apparatus is performing measurements; and a sleep mode when the apparatus is in a non-measurement, minimally powered state,
wherein said controlling comprises operating an energy source management module, operably associated with said controller, to manage voltage in said controller and provide for extended power and low electricity consumption.

15. The method of claim 14, wherein step (c) further comprises controlling operation of the apparatus between said active mode, said sleep mode and a turbo-mode, which is employed in the event that measurement of the water-quality parameter is outside a given range.

16. The method of claim 14, wherein step (b) comprises the chlorine sensor measuring free chlorine or total chlorine.

17. The method of claim 14, wherein step (b) comprises measuring a plurality of water-quality parameters a single liquid sample.

18. The method of claim 14, wherein step (c) further comprises the controller maintaining low power to a Total Chlorine and/or Free Chlorine sensor, so that the sensor remains in an active mode.

19. The method of claim 14, wherein step (c) further comprises the controller providing an alert when a measurement is outside a predetermined range.

20. The method of claim 19, wherein step (c) further comprises the controller disconnecting said at least one sensor if said alert is indicative of a water flow value at or below a predetermined value.

21. The method of claim 14, wherein step (c) further comprises the controller connecting said at least one sensor after a predetermined period of time.

22. The method of claim 14, wherein step (b) comprises detecting illumination from said liquid sample at a 90-degree angle with respect to an illumination beam generated by an illuminator and impinging on said liquid sample, thereby measuring turbidity of the sample.

23. The method of claim 14 wherein step (b) comprises first performing a turbidity measurement, prior to any other measurements.

24. The method of claim 14 wherein step (b) further comprises determining a temperature compensation using an illumination detector disposed at a 180-degree angle to the illumination beam in order to measure the illumination beam.
